(19) United States
(12) Patent Application Publication
(10) Pub. No.: US 2015/0182675 A1
(43) Pub. Date: Jul. 2, 2015

(54) Ascorbic Acid-Eluting Implantable Medical Devices, Systems, and Related Methods

(71) Applicant: South Dakota Board of Regents, Pierre, SD (US)

(72) Inventor: Gopinath Mani, Sioux Falls, SD (US)

(21) Appl. No.: 14/418,221

(22) PCT Filed: Jul. 23, 2013
(86) PCT No.: PCT/US13/51614

§ 371 Date: Jan. 29, 2015

Related U.S. Application Data
(60) Provisional application No. 61/679,958, filed on Aug. 6, 2012.
(60) Provisional application No. 61/834,179, filed on Jun. 12, 2013.

(51) Int. Cl. A61L 31/16; A61M 25/10; A61L 29/16; A61L 29/10; A61L 31/02; A61L 31/08

(52) U.S. Cl. CPC A61L 31/16; A61L 31/022; A61L 31/086; A61L 29/16; A61L 29/10; A61M 25/1029; A61L 2300/428; A61L 2300/21; A61L 2300/412; A61L 2300/606; A61M 2025/1031; A61M 2025/105

(57) ABSTRACT

Implantable medical devices may elute drugs that promote the growth of endothelial cells while inhibiting the growth of smooth muscle cells. In some instances, implantable medical devices may elute L-ascorbic acid, or vitamin C. In some instances, an implantable medical device configured to elute L-ascorbic acid may be a stent, although a variety of other implantable medical devices are contemplated.

### CROSS-REFERENCE TO RELATED APPLICATIONS

This application claims priority from U.S. Provisional Application No. 61/679,958, filed Aug. 6, 2012, and entitled “Ascorbic Acid-Eluting Implantable Medical Devices,” and further claims priority from U.S. Provisional Application No. 61/834,179, filed Jun. 12, 2013, and entitled Ascorbic Acid-Eluting Implantable Medical Devices, Systems, and Related Methods,” both of which are hereby incorporated herein by reference in their entireties.

### FIELD OF THE INVENTION

The application pertains generally to implantable medical devices and more particularly to implantable medical devices that elute therapeutic agents such as ascorbic acid.

### BACKGROUND OF THE INVENTION

Coronary artery disease (CAD) is the leading cause of death in the United States for both men and women. This disease is caused by atherosclerosis, which is a condition that occurs when the arteries are narrowed due to the buildup of atherosclerotic plaque. Percutaneous transluminal coronary angioplasty (PTCA) is frequently performed to open blocked coronary arteries caused by CAD. However, restenosis (arterial re-narrowing) after PTCA was a major limitation and required second revascularization procedure in 30-40% of the patients. Implantation of metal stents reopened the narrowed arteries and provided scaffolding which eliminates vessel recoil and negative remodeling (vessel shrinkage). However, in-stent restenosis because of neo-intima (new tissue) formation remains a significant problem. Drug-eluting stents, which release anti-proliferative drugs (such as, for example, paclitaxel and/or sirolimus) for localized delivery, are a major advancement in the evolution of stents. The anti-proliferative drugs inhibit growth of smooth muscle cells and thereby inhibit in-stent restenosis. However, in some instances, there has been late stent thrombosis in patients having drug eluting stents. Late stent thrombosis is the formation of one or more blood clots in the arteries.

In some instances, the anti-proliferative drugs released from stents can delay or impair re-endothelialization, and this impairment is considered to be a major contributing factor for late stent thrombosis. Most drug-eluting stents in the market are coated with anti-proliferative drugs for treating neointimal hyperplasia. These anti-proliferative drugs are not cell specific; hence, these drugs not only inhibit the growth of smooth muscle cells but also endothelial cells.

Autopsy studies of human FDA approved drug eluting stent implanted coronary arteries suggest that complications of late stent thrombosis are associated with incomplete endothelial coverage of struts. A study compared the endothelialization of a bare metal stent, a sirolimus eluting stent, and a paclitaxel eluting stent in rabbit iliac arteries after 14 days of stent implantation. Poor endothelialization (no struts were endothelialized) was observed for both of the anti-proliferative drug releasing stents which could potentially lead to late stent thrombosis. Re-endothelialization of stent surface is crucial for its long-term success since the endothelial cell lining prevents the adhesion and aggregation of blood platelets and thereby inhibits late stent thrombosis. Hence, there is a need to deliver drugs which promotes the growth of endothelial cells while inhibiting the growth of smooth muscle cells.

### BRIEF SUMMARY OF THE INVENTION

Implantable medical devices may elute drugs that promote the growth of endothelial cells while inhibiting the growth of smooth muscle cells. In some embodiments, implantable medical devices may elute L-ascorbic acid, or vitamin C. In some embodiments, an implantable medical device configured to elute L-ascorbic acid may be a stent, although a variety of other implantable medical devices are contemplated.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a schematic illustration of an implantable medical device in accordance with embodiments of the disclosure.

FIG. 2 is a schematic illustration of a hydroxylated Co-Cr alloy surface in accordance with embodiments of the disclosure.

FIG. 3 is a schematic illustration of an L-AA coating on a hydroxylated Co—Cr alloy surface in accordance with embodiments of the disclosure.

FIG. 4 provides fluorescence microscopy images of FDA stained endothelial cells on days one, three, five, and seven as described in Example One.

FIG. 5 is a graphical representation of resazurin fluorometric assays as described in Example Two.

FIG. 6A is a graphical representation of contact angle data as described in Example Three.

FIG. 6B-1 provides a contact angle image of a control Co—Cr alloy surface without any coating, as described in Example Three.

FIG. 6B-2 provides a contact angle image of a PA coated Co—Cr alloy surface, as described in Example Three.

FIG. 6B-3 provides a contact angle image of a L-AA coated Co—Cr alloy surface, as described in Example Three.

FIG. 7A provides an SEM image (at 27×) of L-AA coated onto a hydroxylated Co—Cr alloy surface as described in Example Four.

FIG. 7B provides an SEM image (at 200×) of L-AA coated onto a hydroxylated Co—Cr alloy surface as described in Example Four.

FIG. 7C provides an SEM image (at 500×) of L-AA coated onto a hydroxylated Co—Cr alloy surface as described in Example Four.

FIG. 7D provides an SEM image (at 1,000×) of L-AA coated onto a hydroxylated Co—Cr alloy surface as described in Example Four.

FIG. 8A provides an SEM image (at 700×) of L-AA coated onto a hydroxylated Co—Cr alloy coronary stent surface as described in Example Four.

FIG. 8B provides an SEM image (at 1,000×) of L-AA coated onto a hydroxylated Co—Cr alloy coronary stent surface as described in Example Four.

FIG. 8C provides an SEM image (at 5,000×) of L-AA coated onto a hydroxylated Co—Cr alloy coronary stent surface as described in Example Four.

FIG. 8D provides an SEM image (at 10,000×) of L-AA coated onto a hydroxylated Co—Cr alloy coronary stent surface as described in Example Four.

FIG. 9A-1 provides an optical profilometer image of a chemically cleaned Co—Cr alloy surface (the control) as described in Example Four.

FIG. 9A-2 provides an optical profilometer image of a phosphoric acid coated Co—Cr alloy surface as described in Example Four.

FIG. 9A-3 provides an optical profilometer image of a phosphoric acid coated and L-AA deposited Co—Cr alloy surface as described in Example Four.

FIG. 9B-1 provides an AFM image of the specimen depicted in FIG. 9A-1 and as described in Example Four.

FIG. 9B-2 provides an AFM image of the specimen depicted in FIG. 9A-2 and as described in Example Four.

FIG. 9B-3 provides an AFM image of the specimen depicted in FIG. 9A-3 and as described in Example Four.

FIG. 10 is a graphical representation of FTIR data as described in Example Four.

FIG. 11 is a graphical representation of elution data as described in Example Five.

FIG. 12A provides a phase contrast microscopy image of day 7 of an endothelial cell culture to which nothing has been added (control), as described in Example Six.

FIG. 12B provides a phase contrast microscopy image of day 7 of an endothelial cell culture to which sirolimus has been added, as described in Example Six.

FIG. 12C provides a phase contrast microscopy image of day 7 of an endothelial cell culture to which paclitaxel has been added, as described in Example Six.

FIG. 12D provides a phase contrast microscopy image of day 7 of an endothelial cell culture to which ascorbic acid has been added, as described in Example Six.

FIG. 13 is a graphical representation of resazurin fluorometric assays as described in Example Seven.

FIG. 14 is a graphical representation of resazurin fluorometric assays as described in Example Eight.

FIG. 15 provides fluorescence microscopy images of FDA stained smooth muscle cells on four different days as described in Example Nine.

FIG. 16A provides a phase contrast microscopy image of day 7 of smooth muscle cell growth after nothing has been added to the culture (control), as described in Example Ten.

FIG. 16B provides a phase contrast microscopy image of day 7 of smooth muscle cell growth after sirolimus has been added to the culture, as described in Example Ten.

FIG. 16C provides a phase contrast microscopy image of day 7 of smooth muscle cell growth after paclitaxel has been added to the culture, as described in Example Ten.

FIG. 16D provides a phase contrast microscopy image of day 7 of smooth muscle cell growth after ascorbic acid has been added to the culture, as described in Example Ten.

FIG. 17 is a graphical representation of resazurin fluorometric assays as described in Example Eleven.

FIG. 18 provides fluorescence microscopy images of FDA stained smooth muscle cells on four different days as described in Example Twelve.

FIG. 19A provides an SEM image of a Co—Cr alloy surface with a PLGA coating prior to L-AA incorporation, as described in Example Thirteen.

FIG. 19B provides an SEM image of the Co—Cr surface of FIG. 19A with the PLGA coating after the L-AA was deposited, as described in Example Thirteen.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

L-ascorbic acid is a water soluble molecule with powerful anti-oxidant properties. L-ascorbic acid has the chemical structure shown below:

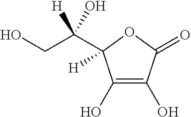 [SMILES: O=C1O[C@H]([C@@H](O)CO)C(O)=C1O]

L-ascorbic acid can be used as a therapeutic agent that limits the oxidation of low density lipoprotein and thereby decreases the risk for coronary artery disease. In addition to its anti-oxidant properties, L-ascorbic acid has significant effect on endothelial cells and smooth muscle cells, both of which are involved in late stent thrombosis and neointimal hyperplasia. L-ascorbic acid promotes the growth of endothelial cells and inhibits the proliferation of smooth muscle cells. In addition, L-ascorbic acid has other significant benefits in inhibiting the growth of macrophages and blood platelets.

In some embodiments, an implantable medical device may be coated with a therapeutic agent that is either L-ascorbic acid or a source or derivative thereof. An illustrative but non-limiting example is ascorbyl palmitate, which is a compound in which ascorbic acid and palmitic acid are connected via an ester bond. Under physiological conditions, the ester bonds break and the ascorbic acid and the palmitic acid are released. Another illustrative but non-limiting example is ascorbyl stearate. Two other examples of ascorbic acid derivates include, but are not limited to, L-Ascorbic acid 2-phosphate sesquimagnesium salt hydrate and 2-Phospho-L-ascorbic acid trisodium salt.

In some embodiments, an implantable medical device may be coated with L-ascorbic acid. In some instances, an implantable medical device may be coated with two or more different therapeutic agents, with one of the therapeutic agents being L-ascorbic acid or a source or derivative thereof. In some embodiments, two or more therapeutic agents may be physically mixed or otherwise combined before being applied to the implantable medical device. In some embodiments, a therapeutic agent such as L-ascorbic acid may be coated onto a stent or other implantable medical device that already includes another therapeutic agent disposed on the device.

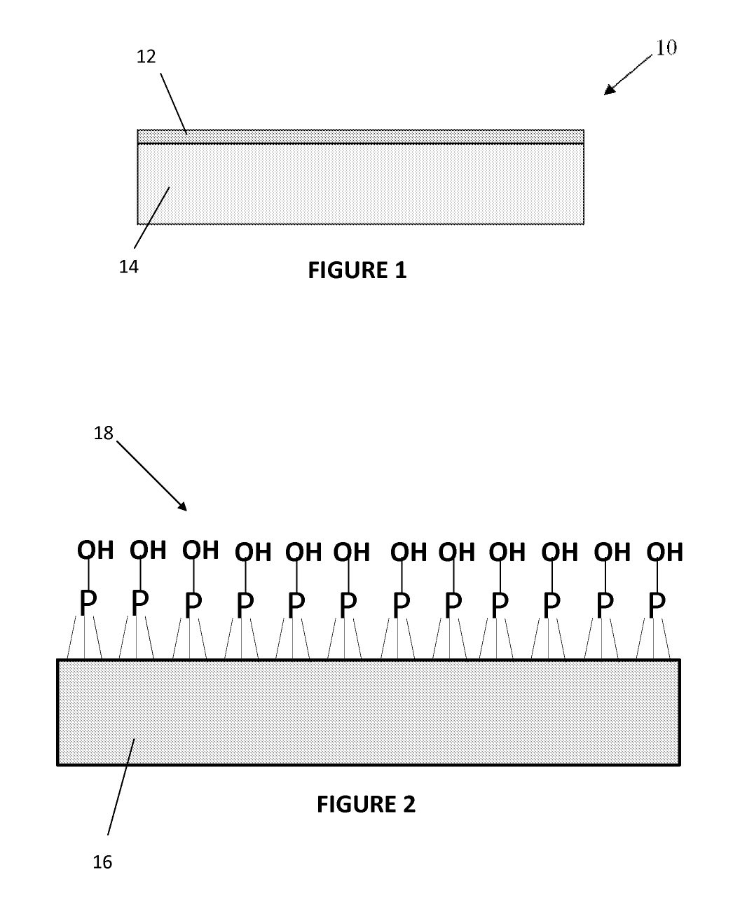

FIG. 1 is a schematic illustration of an implantable medical device 10. The implantable medical device 10 generally includes a surface 12 and a coating 14. The surface 12 of the device 10 may be formed of or otherwise include a variety of metallic, polymeric or ceramic substrates. It will be appreciated that the implantable medical device 10 schematically represents a variety of different implantable medical devices or portions thereof. Illustrative but non-limiting examples of implantable medical devices 10 include cardiovascular devices such as stents, heart valves, artificial hearts, pacemakers, defibrillators, vascular grafts, endovascular stent grafts, transcatheter heart valves, heart assist devices, ventricular assist devices, counterpulsation devices, cardiopulmonary bypass devices, and balloon catheters. Additional devices include orthopedic devices, fracture fixation devices, dental implants, opthomological devices, neural devices, sutures, and tissue engineering scaffolds. In general, any implantable device having a surface to which L-ascorbic acid or a source thereof may be secured is contemplated herein.

In some embodiments, the implantable medical device 10 may be a stent. Stents may be formed of metallic materials, polymeric materials, and/or ceramic materials. Illustrative but non-limiting examples of metallic materials that can be used in devices 10 such as stents include stainless steel, tantalum and tantalum alloys, titanium and titanium alloys including NITINOL, platinum-iridium alloys, magnesium and magnesium alloys and cobalt-chromium alloys.

As shown in FIG. 1, the surface 12 may be an inner surface or an outer surface of the device 10. In some embodiments, the surface 12 may be positioned in or on the implantable medical device 10 such that the surface 12 may be contacted with a bodily fluid and thus provide a mechanism for eluting the coating 14 as bodily fluids are water-based. The surface 12 may be positioned, for example, to be in contact with a bodily fluid such as blood or spinal fluid.

In some embodiments, the coating 14 is the therapeutic agent, such as L-ascorbic acid. Alternatively, the coating 14 may be a mixture or other combination of the therapeutic agent and another material, such as a polymer-based material, a polymer-free material, or some other material that can help to retain the therapeutic agent. According to some embodiments, such a coating 14 could be made by first mixing or otherwise combining the therapeutic agent and the other material(s), and then applying the mixture to the surface 12. Alternatively, the agent and the other material(s) can applied separately and mixed on the surface 12. In a further implementation, the coating 14 may be two or more separate coatings, one of which is the therapeutic agent and the other one or more of which is one or more materials that serve to help retain the agent.

In certain implementations, the coating 14 may be polymer based, in which the L-ascorbic acid is either dispersed within the polymer or bound to the polymer via covalent, ionic, or hydrogen bonding. In some instances, the coating 14 may represent a porous ceramic layer that includes L-ascorbic acid therein. In some embodiments, the coating 14 may be a polymer-free coating. In some instances, the L-ascorbic acid may be disposed on or within a coating 14 that is made up of a microporous surface, a mesoporous metal oxide, a metal organic frame work, a mineral coating, a self-assembled monolayer, or a layer by layer coating.

In certain embodiments, the one or more polymers in the polymer-based coating can include, but are not limited to, poly(lactic-co-glycolic acid (PLGA), dextran, dextran sulfate, polycaprolactone, and polycaprolactone copolymers.

In some embodiments, the L-ascorbic acid may be disposed on or within coating made up of a rough or smooth surface that is then covered with a top coat. The top coat may include one or more of non-polymer materials, including organic or mineral coatings such as dithiothreitol, glutathione, ascorbic acid 6-palmitate, dehydroascorbic acid, acetylsalicylic acid, ethylenediaminetetraacetic acid, sodium phosphate, potassium phosphate, ammonium acetate, octadecylphosphonic acid, 16-phosphonohexadecanoic acid, 11-phosphonoundecanoic acid, 1-dodecylphosphonic acid, phosphoric acid, phosphonoacetic acid, and other alkyl phosphonic acids and alkyl carboxylic acids. Alternatively, the top coat can be made of one or more polymers. In a further alternative, the therapeutic agent can be the coating 14 on the rough or smooth surface 12 of the device 10 and the top coat can cover the agent coating 14.

In some embodiments, the coating 14 may be polymer free. In some instances, the coating 14 may include functional groups bound to the surface 12. Examples of suitable functional groups include but are not limited to hydroxyl groups (—OH), carboxylic acid groups (—COON) and amine groups (—NH₂). L-ascorbic acid may form hydrogen bonds or covalent bonds with these functional groups. It will be appreciated that there are a variety of ways to add these functional groups to the surface 12, depending on the chemical makeup or the structure of the surface 12.

In some embodiments, the surface 12 may be hydroxylated using phosphoric acid, which has the chemical structure shown below:

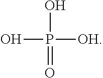

In some embodiments, a hydroxylated surface may be formed on a metallic substrate such as a Co—Cr substrate by immersing the metallic substrate in an aqueous solution containing phosphoric acid according to a known process. In some instances, heating the metallic substrate after immersion can help to stabilize the coating. In some embodiments, the phosphoric acid may form a molecular coating on the metallic substrate. This is illustrated, for example, in FIG. 2.

FIG. 2 is a schematic illustration of a hydroxylated Co—Cr alloy surface 16 after undergoing treatment with phosphoric acid. As illustrated, the phosphoric acid molecules 18 have bonded to the Co—Cr alloy surface 16 via covalent bonding between the oxygen atom and hydroxyl moieties on the phosphoric acid molecule 18 and the Co—Cr alloy surface 16 itself.

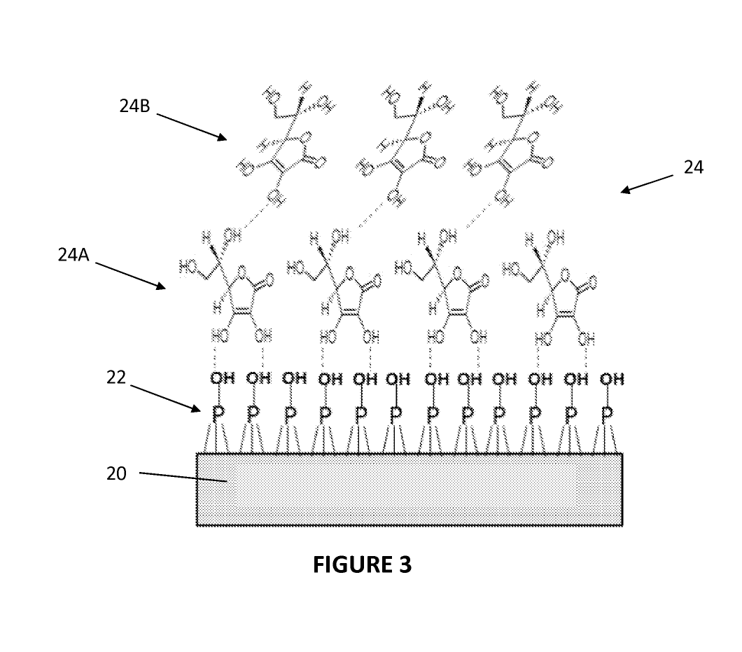

FIG. 3 is a schematic illustration of an L-ascorbic acid coating 24 on a hydroxylated Co—Cr alloy substrate 20 such as that shown in FIG. 2. In FIG. 3, it can be seen that there is a first layer of L-ascorbic acid 24A that forms a molecular coating on the layer of bound phosphoric acid 22. The first layer of L-ascorbic acid 24A hydrogen bonds to the phosphoric acid 22. As illustrated, a second layer of L-ascorbic acid 24B forms atop the first layer of L-ascorbic acid 24A and hydrogen bonds to the first layer of L-ascorbic acid 24A. It will be appreciated that a plurality of layers of L-ascorbic acid may be secured to the Co—Cr alloy substrate 20.

### EXAMPLES

A variety of experiments were carried out to demonstrate the performance of L-AA in encouraging endothelial cell growth as well as adherence and subsequent elution from an implantable medical device.

### Example One

In Example 1, 100 682 g of L-ascorbic acid, 100 μg of sirolimus, and 100 μg paclitaxel were added to separate endothelial cell cultures (15,000 cells per well). A control cell culture was also provided in which no agent was added. The endothelial cell adhesion in the culture well was investigated by staining the live cells with fluorescein diacetate (FDA). The FDA-stained live cells were then imaged using fluorescence microscopy on day one, day 3, day five, and day seven of culture. After five days, a determination of the spreading of endothelial cells was made. After seven days, a determination of growth (viability and proliferation) of endothelial cells was made.

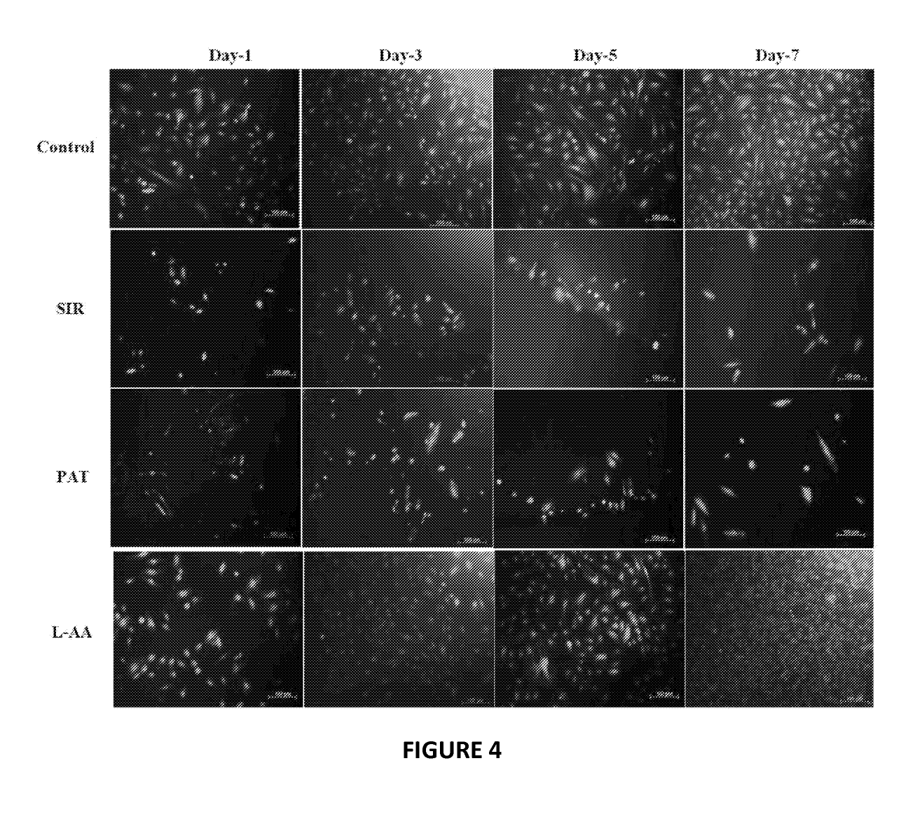

FIG. 4 provides the results. On day 1 (the first column), the endothelial cell adhesion was excellent for ascorbic acid and poor for sirolimus and paclitaxel, while the control exhibited cell adhesion as well. The number of viable endothelial cells in the culture wells containing ascorbic acid was significantly greater than that of the culture wells containing sirolimus and paclitaxel. These results demonstrate that ascorbic acid strongly encourages the endothelial cell adhesion when compared to sirolimus and paclitaxel.

Endothelial cell spreading is an important parameter to analyze since it is directly related to the endothelialization of stents. On day 5, spreading of cells in the culture wells treated with ascorbic acid was excellent while spreading was poor in the culture wells treated with sirolimus and paclitaxel. Most of the cells maintain a round shape in the culture wells treated with sirolimus and paclitaxel. The control exhibited some spreading. These results demonstrate that the ascorbic acid strongly encourages the spreading of endothelial cells when compared to the other antiproliferative drugs such as sirolimus and paclitaxel.

On day 7, the growth (viability and proliferation) of endothelial cells treated with ascorbic acid was excellent while the growth was poor for the cells treated with sirolimus and paclitaxel. The cells treated with L-AA exhibited more growth in comparison to the control. The number of viable endothelial cells on ascorbic acid was significantly greater than that of sirolimus and paclitaxel after 7 days of culture. These results demonstrate that the ascorbic acid strongly encourages the growth of endothelial cells when compared to sirolimus and paclitaxel.

### Example Two

In order to provide a quantitative characterization of the Example One results, a resazurin fluorometric assay (Alamar Blue) was used. A solution of Alamar Blue was added to the endothelial cell cultures which were already treated with ascorbic acid, sirolimus, or paclitaxel. A control lacking any therapeutic agent was included. After respective time points (1, 3, 5, and 7 days), the fluorescence of the solution was measured using a microplate reader. The collected data is provided as relative fluorescence units (RFU) vs. time for all the three groups (ascorbic acid, sirolimus, and paclitaxel) of samples.

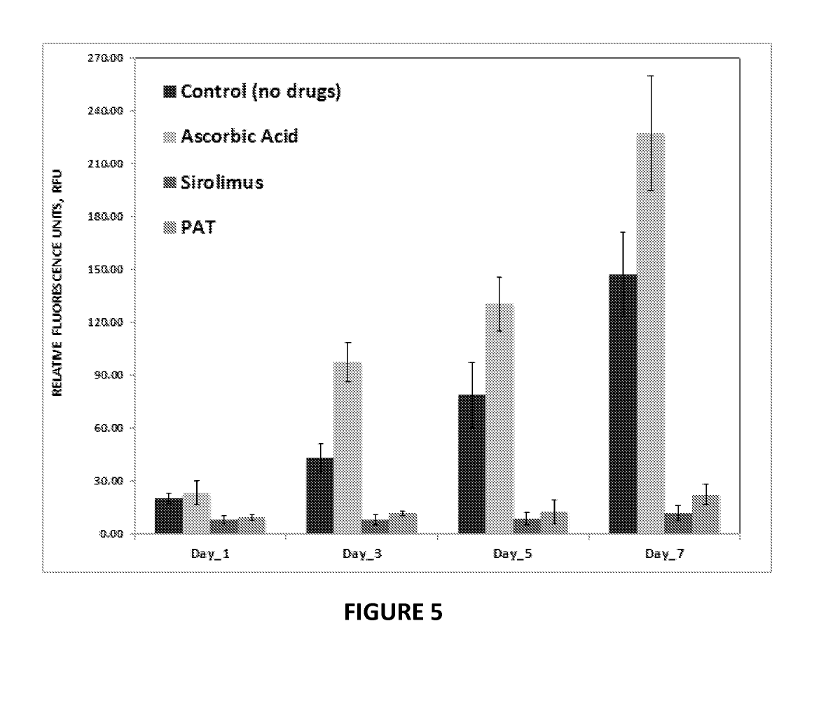

The results are shown in FIG. 5. The growth of endothelial cells treated with ascorbic acid was 19-fold, 10-fold, and 1.6 fold greater than that of the cells treated with sirolimus, paclitaxel, and control (no drug), respectively. This result demonstrates the superiority of L- ascorbic acid for promoting endothelialization over other anti-proliferative drugs currently used in stents.

### Example Three

Attachment of phosphoric acid to a metal substrate was tested using a cobalt-chromium (“Co—Cr”) alloy. Both the control and test specimens were treated by a chemical cleaning procedure to remove any contaminants from the Co—Cr alloy surface. The chemical cleaning procedure was carried out by sonicating the Co—Cr alloy specimens in ethanol, acetone, and methanol twice for 10 min each. The specimens were then dried under nitrogen gas. Then, the test Co—Cr alloy specimens were immersed in a 100 mM solution of phosphoric acid in deionized water (di-H2O) for 24 hours. The test specimens were heated at 120° C. in air for 19 hours followed by cleaning in di-H2O for 1 minute.

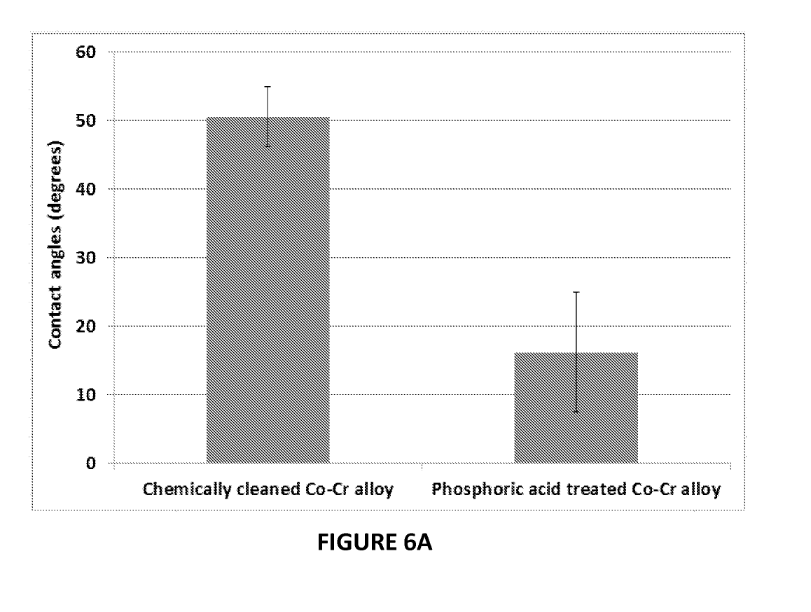

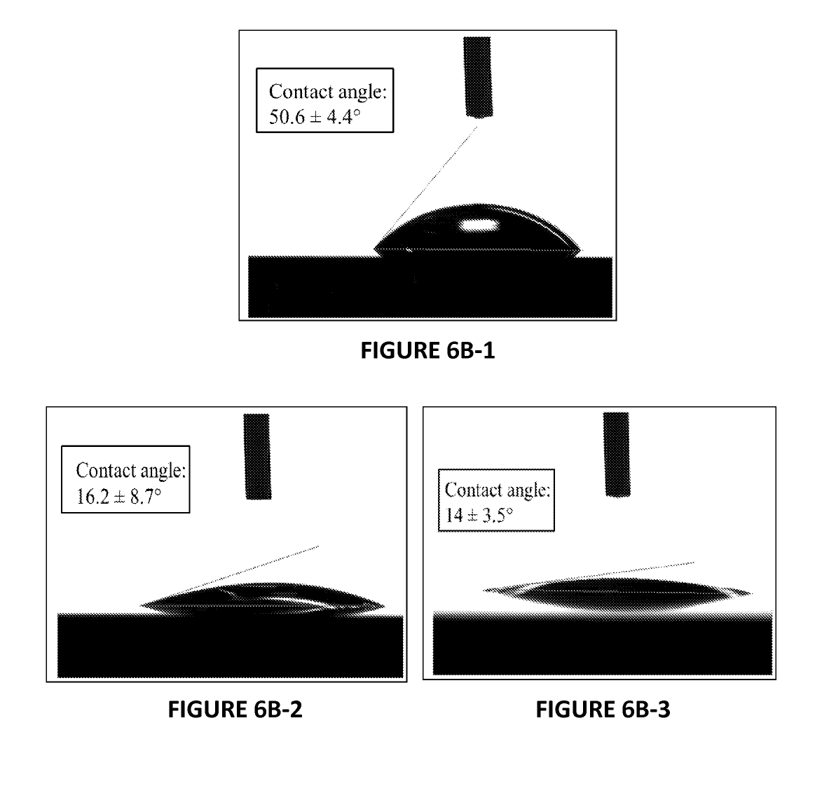

Both the control and test specimens were characterized using contact angle goniometry and the results are shown in graph form in FIG. 6A. For the chemically cleaned control Co—Cr alloy, a contact angle value of 50.6±4.4° was obtained. However, after phosphoric acid treatment, the contact angle value significantly reduced to 16.2±8.7°. This suggests that the phosphoric acid is bound to Co—Cr alloy surfaces and provides a hydroxyl (—OH) group enriched surfaces.

FIGS. 6B-1, 6B-2, and 6B-3 show contact angle images of the control (chemically cleaned Co—Cr alloy) (FIG. 6B-1), the phosphoric acid coated specimen (FIG. 6B-2), and another phosphoric acid coated specimen on which ascorbic acid was deposited (FIG. 6B-3). The phosphoric acid and ascorbic acid deposited specimens exhibited a contact angle values of 16.2±8.7° and 14±3.5°, respectively. This suggests that the phosphoric acid was bound to the Co—Cr alloy surface and that ascorbic acid was successfully deposited thereon.

### Example Four

Attachment of L-ascorbic acid to a hydroxylated Co—Cr substrate was tested. A solution of L-ascorbic acid was prepared in ethanol at a concentration of 4 mg/mL. A 75 μL aliquot of the prepared L-ascorbic acid solution was carefully placed on an hydroxylated Co—Cr alloy surfaces (1 cm×1 cm) using a micropipette. The solution was allowed to evaporate in air at 37° C. for 24 hours leaving behind a thin L-ascorbic acid film on alloy surfaces. The L-ascorbic acid deposited alloy specimens were characterized using scanning electron microscopy (SEM), optical profilometer, and Fourier transform infrared spectroscopy (FTIR).

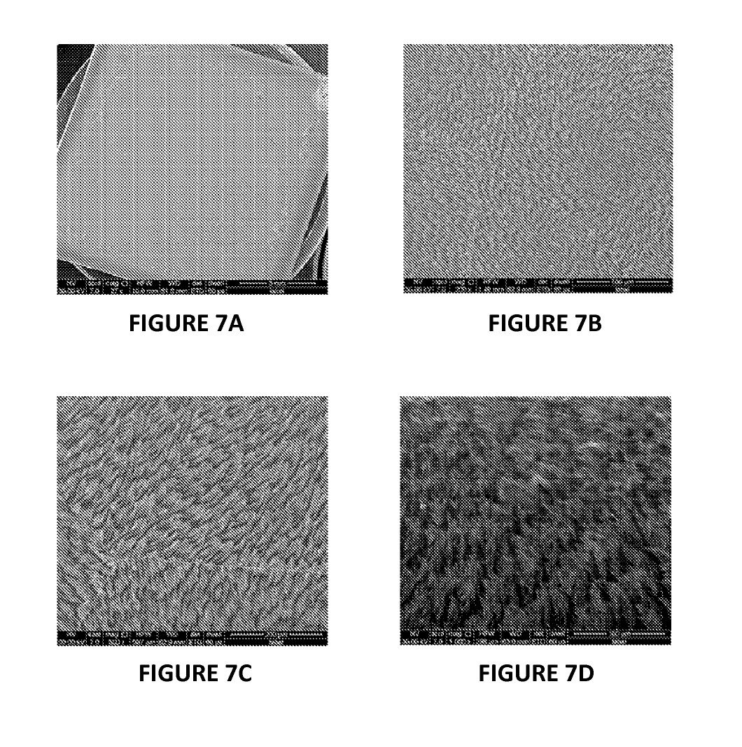

FIGS. 7A-7D include SEM images acquired at 27× (FIG. 7A), 200× (FIG. 7B), 500× (FIG. 7C) and 1,000× (FIG. 7D). These images show the presence of feather shaped ascorbic acid crystals uniformly deposited on Co—Cr alloy surfaces. This result strongly demonstrates the successful coating of ascorbic acid on Co—Cr alloy.

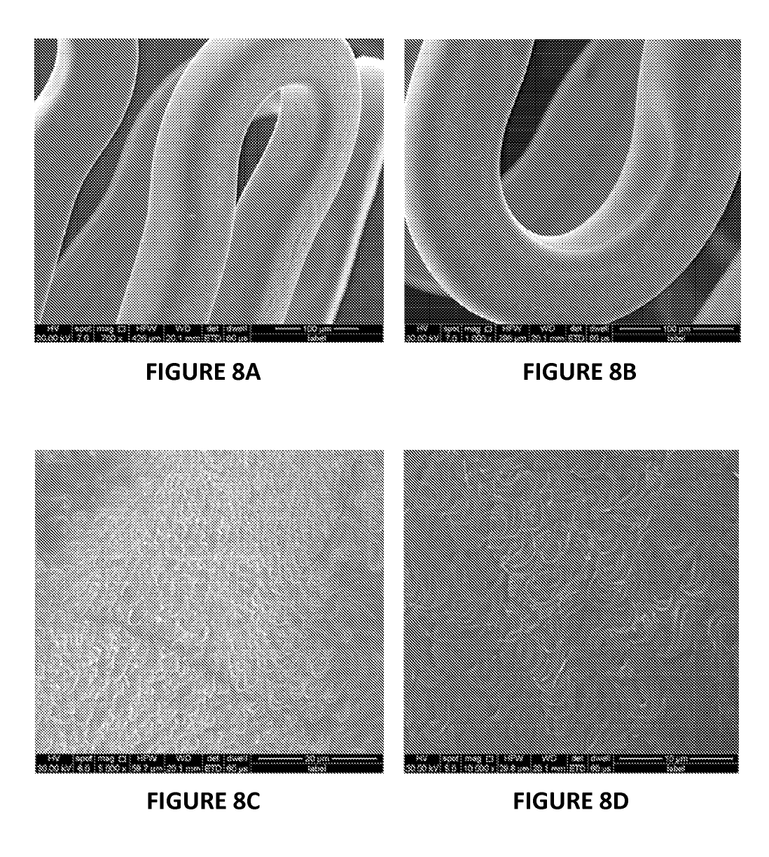

FIGS. 8A-8D include SEM images acquired at 700× (FIG. 8A), 1,000× (FIG. 8B), 5,000× (FIG. 8C), and 10,000× (FIG. 8D). These images show uniform deposition of feather shaped L-ascorbic acid crystals on the stent struts. These results demonstrated the successful deposition of ascorbic acid on 3D cardiovascular stents.

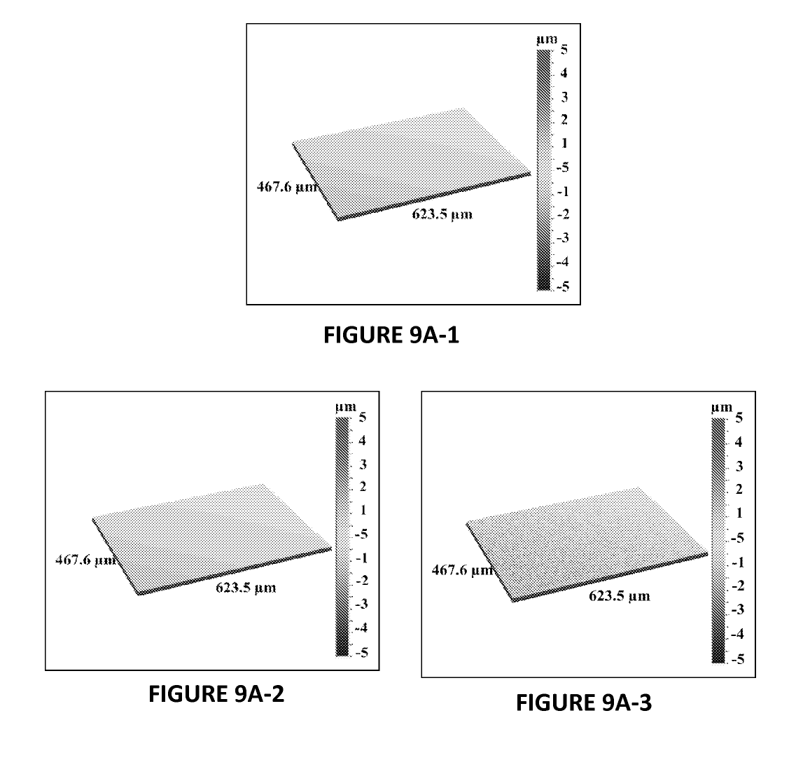

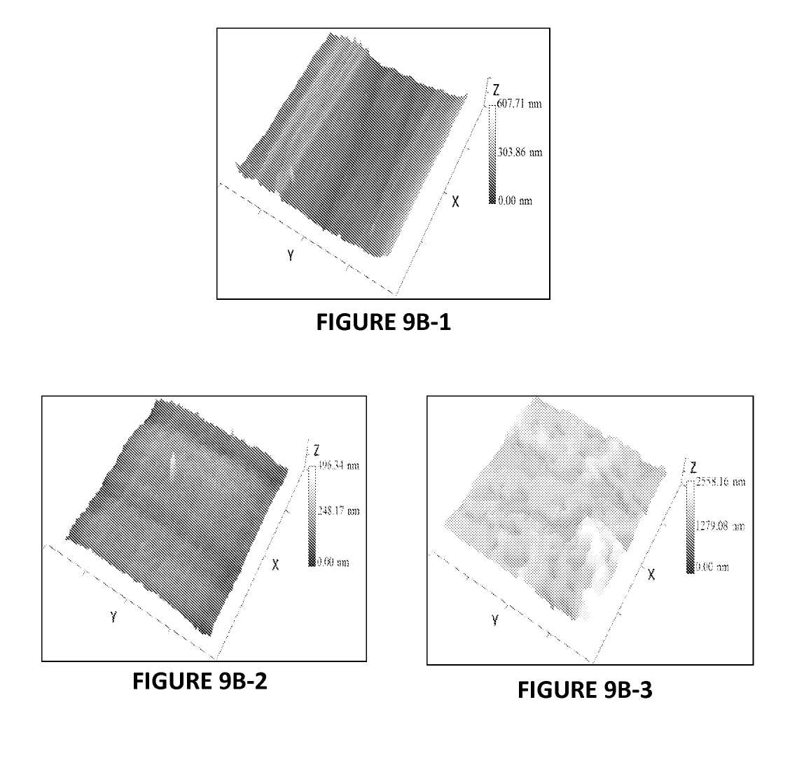

FIGS. 9A-1, 9A-2, and 9A-3 provide optical profilometer images of a chemically-cleaned Co—Cr alloy (the control) (FIG. 9A-1), a Co—Cr alloy coated with phosphoric acid (FIG. 9A-2), and a phosphoric acid coated Co—Cr alloy on which L-ascorbic acid has been deposited (FIG. 9A-3). The optical profiler images showed a flat surface for the control surface (FIG. 9A-1) and for the surface coated with phosphoric acid (FIG. 9A-2). However, after ascorbic acid deposition, feather shaped ascorbic acid crystals were uniformly present on Co—Cr alloy surfaces (FIG. 9A-3). This demonstrates, in agreement with the SEM characterization, the successful deposition of ascorbic acid coating on Co—Cr alloy surfaces.

FIGS. 9B-1, 9B-2, and 9B-3 provide AFM images (scan size=10×10 μm) of the control (FIG. 9B-1), the phosphoric acid coated Co—Cr alloy (FIG. 9B-2), and the phosphoric acid coated and L-ascorbic acid deposited Co—Cr alloy specimens (FIG. 9B-3) discussed above. These images demonstrate, in agreement with the SEM characterization and the optical profilometer images above, the successful deposition of ascorbic acid coating on Co—Cr alloy surfaces.

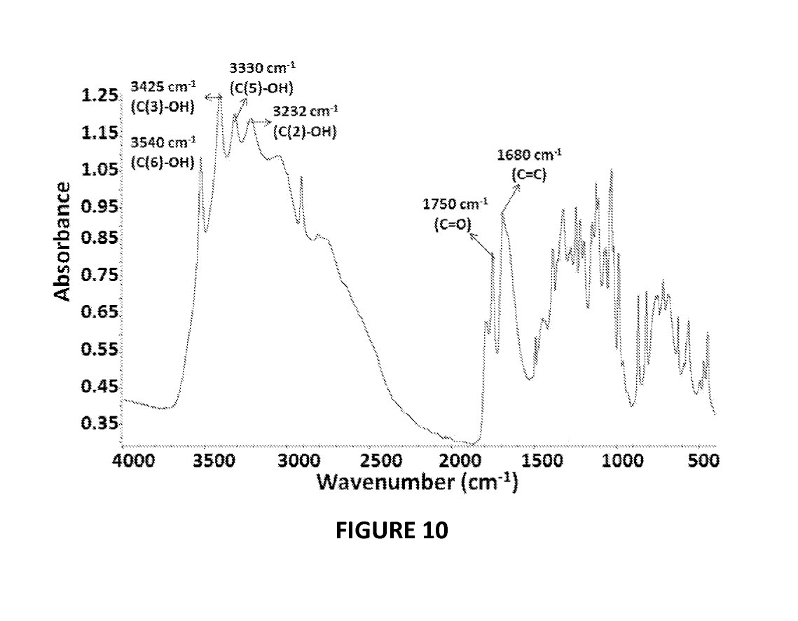

FIG. 10 provides the FTIR spectrum, showing strong peaks for the four-OH groups: groups: C(2)—OH at 3232 cm⁻¹; C(5)—OH at 3330 cm⁻¹; C(3)—OH at 3425 cm⁻¹; and C(6)—OH at 3540 cm⁻¹. The peaks for C═O (1750 cm⁻¹) and C═C (1680 cm⁻¹) bonds were also present. These results successfully demonstrated the coating of ascorbic acid on Co—Cr alloy surfaces.

### Example Five

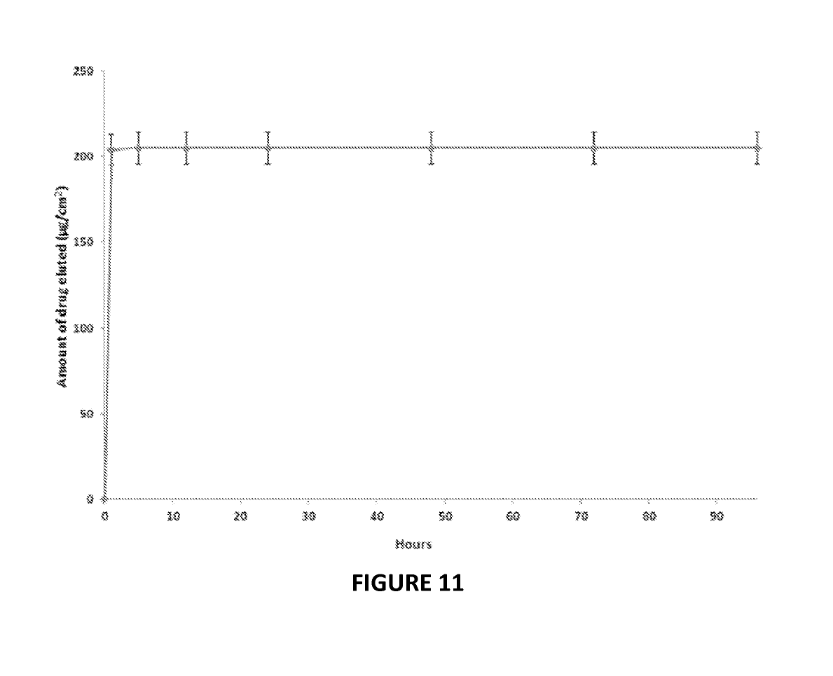

A drug release study was performed. L-ascorbic acid coated Co—Cr alloy specimens were immersed in tris-buffered saline (TBS) at 37° C. for up to 4 days. The TBS solution was collected at pre-determined time points and analyzed for the amount of L-ascorbic acid released using high performance liquid chromatography (HPLC). The results are shown in FIG. 11, which indicates that ascorbic acid was successfully delivered from Co—Cr alloy surfaces.

### Example Six

In Example 6, 100 μg of L-ascorbic acid, 100 μg of sirolimus, and 100 μg paclitaxel were added to separate endothelial cell cultures. A control cell culture was also provided in which no agent was added. The effects of L-AA, SIR, and PAT on the growth of endothelial cells was investigated by taking phase contrast images of cells after 7 days using an Axiovert 200 M inverted microscopy (Carl Zeiss) in the bright field imaging mode and examining the cell morphology.

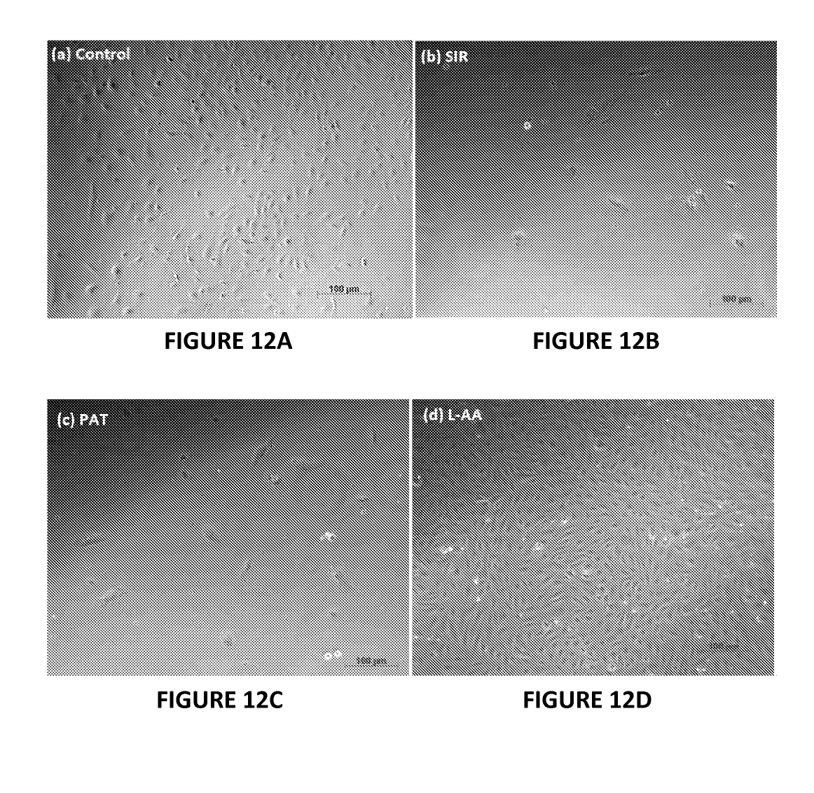

FIGS. 12A-12D provide the day 7 results. The endothelial cells showed spreading morphology with characteristic polygonal shape for the control (FIG. 12A) and L-AA (FIG. 12D), while showing uncharacteristic oval or round shape with no spreading morphology for SIR (FIG. 12B) and PAT (FIG. 12C). Thus, the results showed that the characteristic morphological features of endothelial cells were well maintained for L-AA and the control while such features were not present in the cells treated with SIR or PAT. These results demonstrate that ascorbic acid strongly encourages endothelial cell growth when compared to sirolimus and paclitaxel.

### Example Seven

In Example 7, a quantitative resazurin fluorometric assay was used. A solution of alamarBlue® from a kit purchased from Biotium Inc. (in Hayward, Calif.) was added to the endothelial cell cultures which were already treated with different doses of ascorbic acid. More specifically, the different doses of ascorbic acid included 1 μg/mL, 100 μg/mL, 300 μg/mL, 500 μg/mL, and 1000 μg/mL. A control lacking any ascorbic acid was also included. After respective time points (1, 3, 5, and 7 days), the fluorescence of the solution was measured using a microplate reader. The collected data is provided as relative fluorescence units (RFU) vs. time for all the six groups (control, 1 μg/mL, 100 μg/mL, 300 μg/mL, 500 μg/mL, and 1000 μg/mL) of samples.

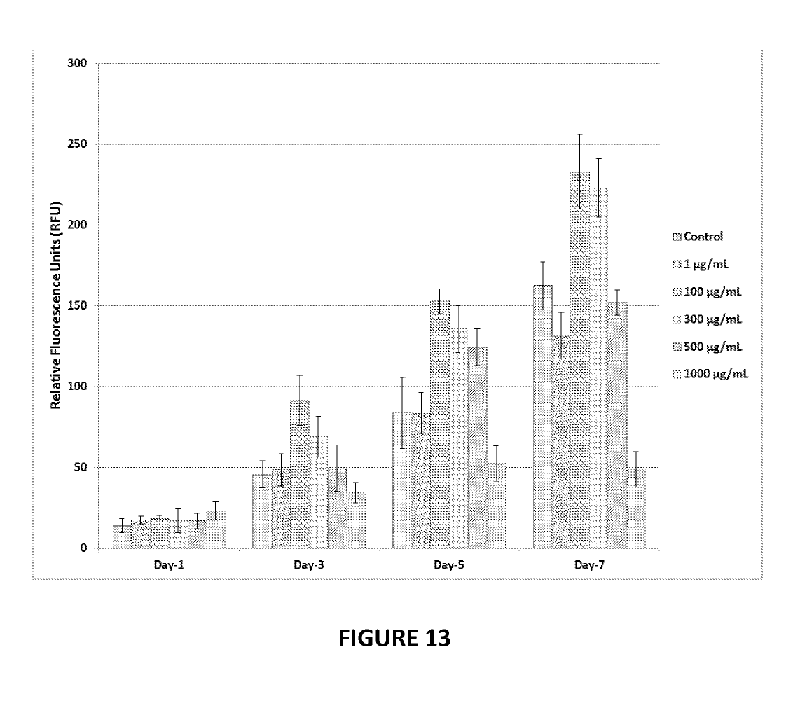

The results are shown in FIG. 13. On day 1, no significant differences in the number of cells were observed. On day 3, the number of cells observed for the 100 μg dose of ascorbic acid was significantly greater than that of the control or the other ascorbic acid doses except the 300 μg dose. On day 5, the ascorbic acid doses of 100, 300, and 500 μg showed significantly greater number of cells when compared to that of the control and the other doses (1 and 1000 μg). On day 7, the doses 100 and 300 pg showed a maximum number of cells among the different groups and were significantly greater than that of the control and the other doses. Based on these results, the viability and proliferation of endothelial cells for the different doses of ascorbic acid increased in the following order: 1000 μg<1 μg=control=500 μg<<300 μg=100 μg.

### Example Eight

In Example 8, a quantitative resazurin fluorometric assay was used. A solution of alamarBlue® from a kit purchased from Biotium Inc. (in Hayward, Calif.) was added to the smooth muscle cell cultures which were already treated with ascorbic acid, sirolimus, or paclitaxel. A control lacking any therapeutic agent was also included. After respective time points (1, 3, 5, and 7 days), the fluorescence of the solution was measured using a microplate reader. The collected data is provided as relative fluorescence units (RFU) vs. time for all the three groups (ascorbic acid, sirolimus, and paclitaxel) of samples.

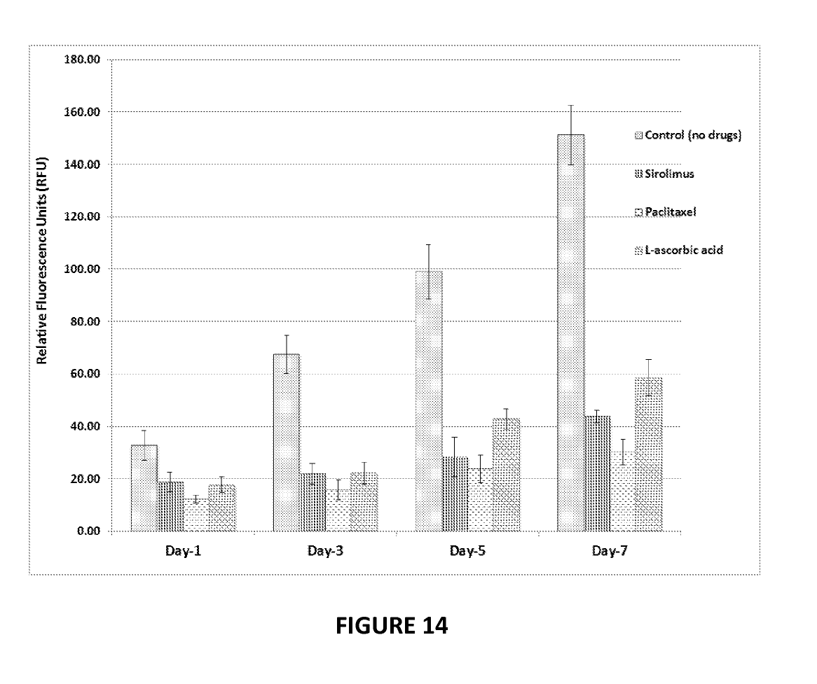

The results are shown in FIG. 14. On day 1, all three treatments showed significantly lesser number of cells in comparison to the control. On day 3, the three treatments significantly inhibited the cell growth and showed lesser number of cells when compared to that of the control, and no significant difference in the number of cells was observed among ascorbic acid, paclitaxel, and sirolimus. A similar trend was observed on day 5, but the sirolimus and paclitaxel treatments showed lesser numbers of cells than that of the ascorbic acid treatment. Similar results were observed on day 7. Based on these results, the SMC viability and proliferation decreased in the following order: Control>>ascorbic acid>sirolimus=paclitaxel. Thus, these results demonstrated that ascorbic acid significantly inhibited growth of SMCs, although the inhibitory effect was inferior to that of sirolimus and paclitaxel.

### Example Nine

Example 9 provides a qualitative characterization of the Example Eight results using flurorescence microscopy. More specifically, the growth of smooth muscle cells in the presence of three different treatments (ascorbic acid, paclitaxel, and sirolimus) was investigated by staining the live cells with fluorescein diacetate (FDA). The FDA-stained live cells were then imaged using flurorescence microscopy on day 1, day 3, day 5, and day 7 of culture. On each day, a determination of growth (viability and proliferation) of smooth muscle cells was made.

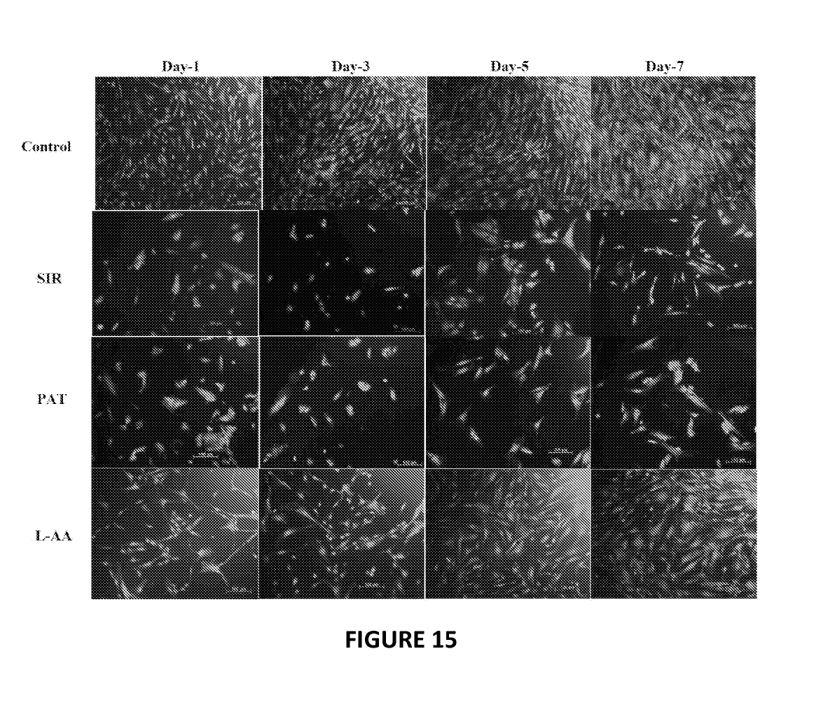

The results are shown in FIG. 15. These images show that the smooth muscle cells were significantly proliferated on the control from one time point to the other while the cell growth was significantly inhibited for ascorbic acid, paclitaxel, and sirolimus. After 7 days, the control sample showed >90% confluence while the ascorbic acid-treated cells showed about 50-60% confluence. Paclitaxel and sirolimus showed very few viable cells with about 20% confluence. These qualitative results are in agreement with the quantitative assessment provided in Example 8.

### Example Ten

Example 10 provides a further qualitative characterization of the Example Eight results using phase contrast microscopy. That is, the effects of L-AA, SIR, and PAT on the growth of smooth muscle cells was investigated by taking phase contrast images of cells after 7 days using an Axiovert 200 M inverted microscopy (Carl Zeiss) in the bright field imaging mode and examining the cell morphology.

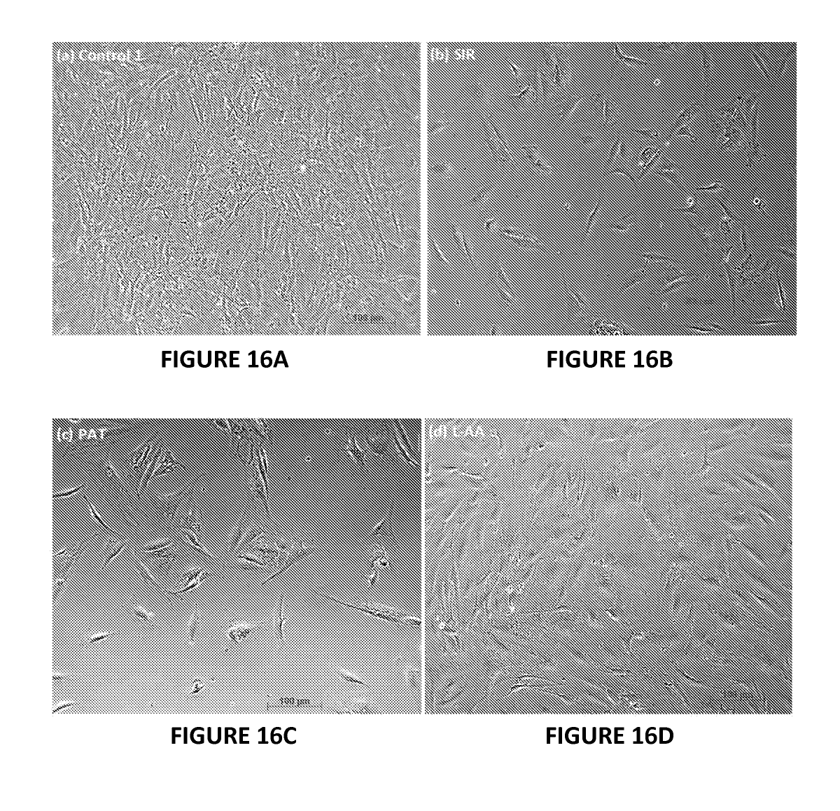

FIGS. 16A-16D provide the day 7 results. The smooth muscle cells showed spreading morphology with characteristic spindle shape for the control (FIG. 16A). For the ascorbic acid (FIG. 16D), the cells were spindle-shaped, but they were less spreading when compared to the control. For paclitaxel (FIG. 16C) and sirolimus (FIG. 16B), the cells were not spreading and only very few cells were spindle-shaped, with the remaining cells being either triangular or irregular-shaped. These results suggest that the spreading of smooth muscle cells was affected by treating with ascorbic acid, sirolimus, and paclitaxel, while the morphological features of cells were also affected by sirolimus and paclitaxel treatments.

### Example Eleven

In Example 11, a quantitative resazurin fluorometric assay was used. A solution of alamarBlue® from a kit purchased from Biotium Inc. (in Hayward, Calif.) was added to smooth muscle cell cultures which were already treated with different doses of ascorbic acid. More specifically, the different doses of ascorbic acid included 1 μg/mL, 100 μg/mL, 300 μg/mL, 500 μg/mL, and 1000 μg/mL. A control lacking any ascorbic acid was also included. After respective time points (1, 3, 5, and 7 days), the fluorescence of the solution was measured using a microplate reader. The collected data is provided as relative fluorescence units (RFU) vs. time for all the six groups (control, 1 μg/mL, 100 μg/mL, 300 μg/mL, 500 μg/mL, and 1000 μg/mL) of samples.

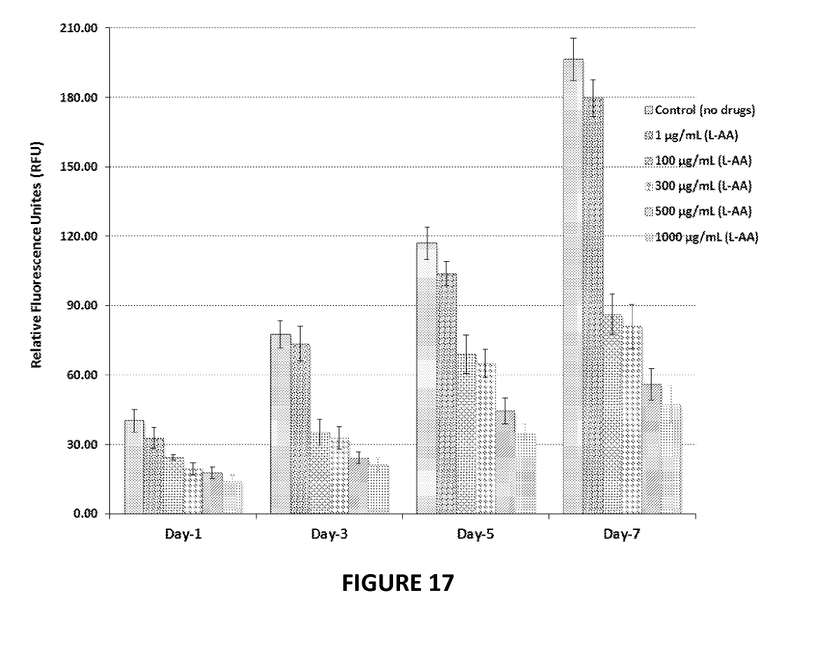

The results are shown in FIG. 17. On day 1, the ascorbic acid doses ranging from 100 to 1000 μg showed significantly fewer cells compared to both the control and the 1 μg ascorbic acid dose. A similar trend was observed on day 3, along with the 500 and 1000 μg ascorbic acid doses showing fewer cells in comparison to the 100 and 300 μg doses. Similar results were observed on days 5 and 7 as well. Based on these results, the viability and proliferation of smooth muscle cells for the different doses of ascorbic acid decreased in the following order: control=1 μg>>100 μg=300 μg>500 μg=1000 μg.

### Example Twelve

Example 12 provides a qualitative characterization of the Example Eleven results using fluorescence microscopy. More specifically, the growth of smooth muscle cells in the presence of five different doses of ascorbic acid was investigated by staining the live cells with fluorescein diacetate (FDA). The FDA-stained live cells were then imaged using fluorescence microscopy on day 1, day 3, day 5, and day 7 of culture. On each day, a determination of growth (viability and proliferation) of smooth muscle cells was made.

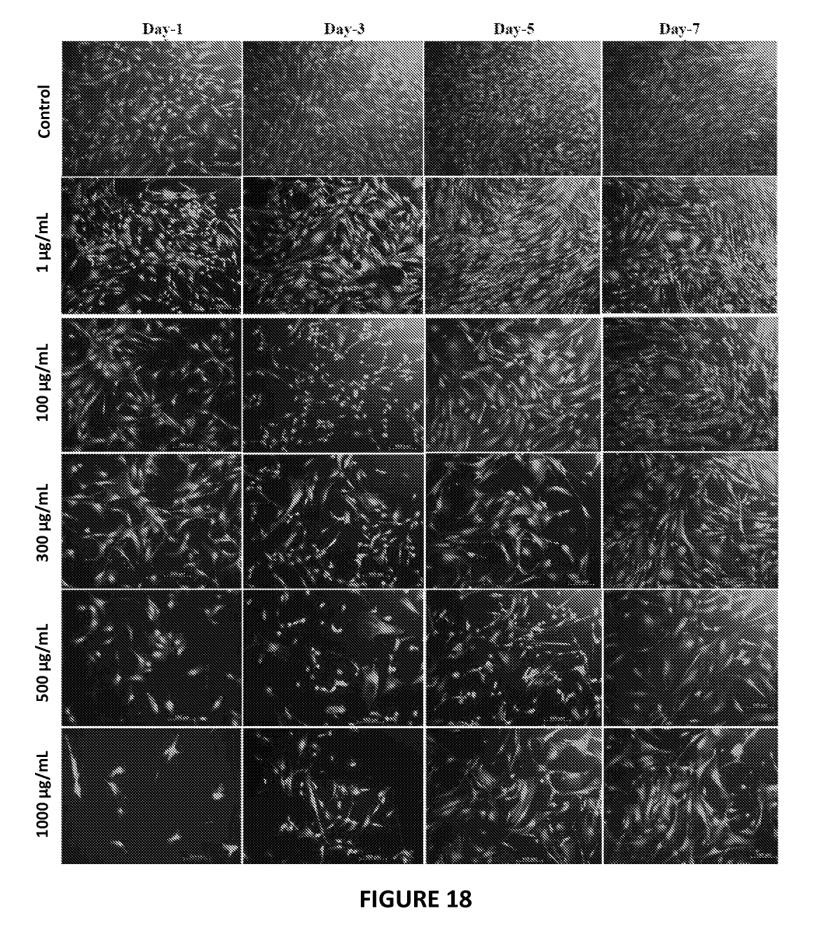

The results are shown in FIG. 18. These images show that the smooth muscle cells were proliferating significantly for the control and the 1 μg dose from one time point to the other while the cell growth was significantly inhibited for the ascorbic acid doses ranging from 100 to 1000 μg. After 7 days, the control sample and the 1 μg dose showed >90% confluence while the 100 and 300 μg doses showed about 50-60% confluence, and the 500 and 1000 μg doses showed about 30-40% confluence. These results demonstrate that ascorbic acid showed dose-dependent inhibitory effect with 1 μg dose showing no inhibitory effect and 1000 μg dose showing maximum inhibitory effect.

Example Thirteen

In Example 13, the use of a polymer-based coating made of poly(lactic-co-glycolic acid (PLGA) on a Co—Cr alloy surface and the deposit of L-AA on the coating was investigated. The PLGA coating was applied to the surface and then the L-AA was deposited.

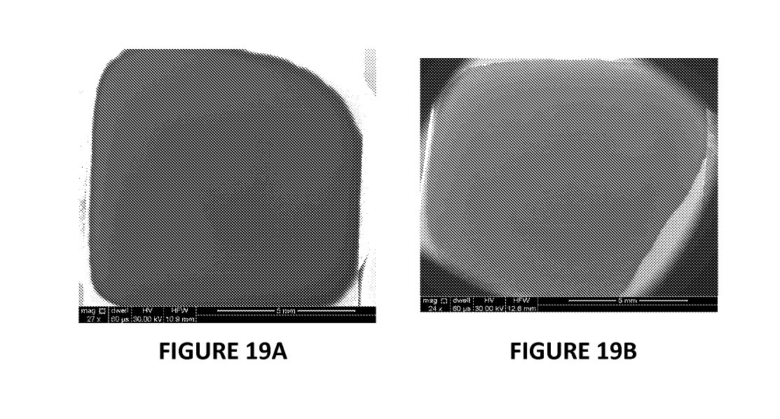

FIGS. 19A-19B include SEM images of a Co—Cr alloy surface with a PLGA coating (prior to L-AA incorporation) (FIG. 19A) and the same Co—Cr surface with the PLGA coating after the L-AA was deposited (FIG. 19B). FIG. 19B shows the presence of ascorbic acid crystals uniformly deposited on the PLGA coating on the Co—Cr alloy surface. This result demonstrates the successful deposit of ascorbic acid on the PLGA coating on the Co—Cr alloy.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

Although the present invention has been described with reference to preferred embodiments, persons skilled in the art will recognize that changes may be made in form and detail without departing from the spirit and scope of the invention.

What is claimed is:

1. An implantable medical device comprising:
a surface; and
a source of ascorbic acid secured relative to the surface.

2. The implantable medical device of claim 1, comprising a device selected from the group consisting of a cardiovascular device, an orthopedic device, a fracture fixation device, a dental implant, an opthomological device, a neural device, a suture and a tissue engineering scaffold.

3. The implantable medical device of claim 1, wherein the surface comprises a functionalized surface to which the ascorbic acid source bonds.

4. The implantable medical device of claim 3, wherein the functionalized surface comprises hydroxyl moieties, carboxylic moieties or amine moieties, and the ascorbic acid bonds to the functionalized surface via covalent bonding or hydrogen bonding.

5. The implantable medical device of claim 1, wherein the ascorbic acid is disposed within a polymer or a ceramic material that is coated onto the surface.

6. An implantable stent comprising:
a stent body having a surface; and
a source of ascorbic acid secured relative to the surface.

7. The implantable stent of claim 6, comprising a metallic stent body, a polymer stent body or a ceramic stent body.

8. The implantable stent of claim 6, wherein the surface comprises a functionalized surface to which the ascorbic acid source bonds.

9. The implantable stent of claim 8, wherein the functionalized surface comprises hydroxyl moieties, carboxylic moieties or amine moieties, and the ascorbic acid source bonds to the functionalized surface via covalent bonding or hydrogen bonding.

10. The implantable stent of claim 6, further comprising a layer of phosphoric acid bound to the surface.

11. The implantable stent of claim 10, wherein the ascorbic acid is bonded to the layer of phosphoric acid.

12. The implantable stent of claim 6, wherein the stent body comprises a cobalt chromium alloy.

13. A method of forming an ascorbic acid-eluting stent, the method comprising:
providing a stent having a surface;
contacting the surface of the stent with phosphoric acid to form a layer of bound phosphoric acid; and
contacting the layer of bound phosphoric acid with a solution of ascorbic acid;
wherein the ascorbic acid forms bonds with the layer of bound phosphoric acid.

14. The method of claim 13, wherein providing a stent comprises providing a metallic stent, a polymeric stent or a ceramic stent.

15. The method of claim 13, wherein providing a stent comprises providing a cobalt chromium alloy stent.

16. A method of forming an inflatable balloon catheter that elutes ascorbic acid, the method comprising:
providing an inflatable balloon having a surface;
treating the surface of the inflatable balloon to accept an ascorbic acid source; and
contacting the surface with a solution of ascorbic acid to form a layer of ascorbic acid on the surface of the inflatable balloon.
